(19) United States
(12) Patent Application Publication
(10) Pub. No.: US 2012/0073360 A1
(43) Pub. Date: Mar. 29, 2012

(54) METHOD AND SYSTEM FOR SURFACE MODIFICATION OF SUPERADSORBENT MATERIAL FOR IMPROVED ENVIRONMENTAL AND URBAN AIR SAMPLING APPLICATIONS

(75) Inventors: Deborah E. Hunka, Belcamp, MD (US); Christopher L. Rector, Lee's Summit, MO (US); Michael J. Bowers II, Sykesville, MD (US)

(73) Assignee: BAE Systems Information and Electronic Systems Integration Inc., Nashua, NH (US)

(21) Appl. No.: 13/183,492

(22) Filed: Jul. 15, 2011

Related U.S. Application Data
(60) Provisional application No. 61/364,603, filed on Jul. 15, 2010.

(51) Int. Cl. G01N 5/02; C07C 211/00; C07C 209/00

(52) U.S. Cl. 73/73; 564/1

(57) ABSTRACT

A method for providing superadsorption of polar organic compounds using a material system comprising the steps of enhancing adsorption by means of using high surface area and mass transfer rates and decreased reactivity at surface sites attractive to the polar compounds and employing consequence management by maintaining a high rate of adsorptivity combined with high fidelity and accuracy of the material system.

### CROSS-REFERENCE TO RELATED APPLICATIONS

This Application claims rights under 35 USC §119(e) from U.S. Application Ser. No. 61/364,603 filed Jul. 15, 2010; and the U.S. application Ser. No. ______ (Atty. Docket BAEP-1295) filed Jul. 15, 2011, the contents both of which are incorporated herein by reference.

### BACKGROUND OF THE INVENTION

1. Field of the Invention

The present invention relates to providing enhanced adsorption and more particularly to methods for providing enhanced adsorption via high surface area and mass transfer rates.

2. Brief Description of Related Art

Current superadsorbent materials do not provide adequate adsorption of polar compounds, i.e., alcohols, amines, and hydrocarbons containing carboxyl groups. Each of these groups represent a portion of chemicals listed as chemical warfare agents, toxic industrial compounds, toxic industrial materials, and other harmful volatile organic compounds.

The combined act of sampling the air in an environment and subsequently detecting the adsorbed samples is defined as consequence management. The current methods of performing this function do not have any solution that can adsorb a wide variety of polar compounds and/or volatile organic compounds and rapidly desorb those compounds with fidelity and accuracy.

A need exists, therefore, for an improved method for providing enhanced adsorption.

### SUMMARY OF THE INVENTION

The present invention is a method for providing superadsorption of polar organic compounds using a material system comprising the steps of:
(a) enhancing adsorption by means of using high surface area and mass transfer rates and decreased reactivity at surface sites attractive to the polar compounds; and
(b) employing consequence management by maintaining a high rate of adsorptivity combined with high fidelity and accuracy of the material system.

According to the present invention, the modification of the superadsorbent material leads to enhanced performance in adsorption of the classes of compounds listed above, which in turn allows the for 1) the identification of the compounds in the original air sample and 2) the ability to correlate a relative concentration of the analytes to an original concentration. While the surface modification of the material allows for more polar compounds to be adsorbed, the desirable physical properties such as very high surface area and mass transfer rates of the superadsorbent material are retained.

The combined act of sampling the air in an environment and subsequently detecting the adsorbed samples is defined as consequence management. The current methods of performing this function do not have any solution that can adsorb a wide variety of volatile organic compounds and rapidly desorb it with very high fidelity and accuracy.

Those skilled in the art will appreciate that the high rate of adsorptivity combined with high fidelity and accuracy of the material system of the method of this invention provides a solution for consequence management.

### BRIEF DESCRIPTION OF THE DRAWINGS

The present invention is further described with reference to the following drawings wherein:

FIG. 1 is a graph showing modified and unmodified sorbent challenged with polar analytes in a preferred embodiment of the invention; and

FIG. 2 is a graph showing unmodified and modified sorbents challenged with polar compounds in an alternate preferred embodiment of the invention.

### DETAILED DESCRIPTION OF THE PREFERRED EMBODIMENTS

The method and system of the present invention is further defined by the following working examples:

### Example 1

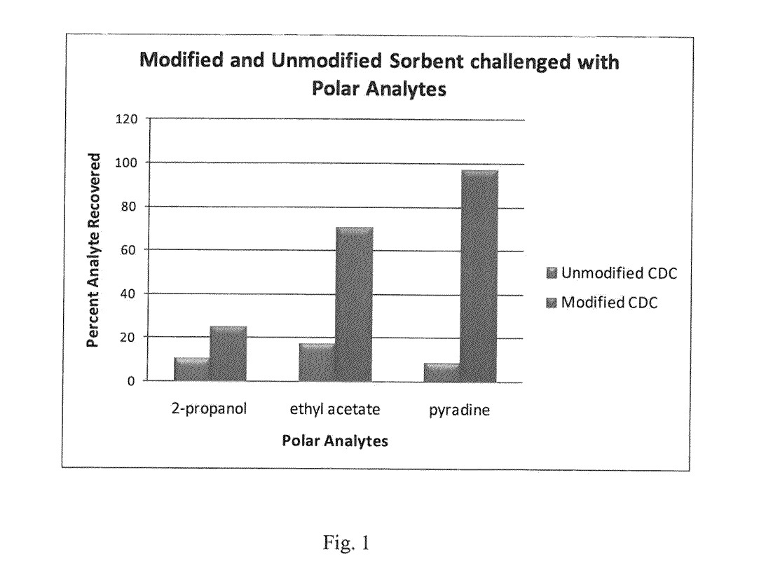

Results in FIG. 1 show the desorption results of an analyte mix with polar compounds from the unmodified sorbent and the sorbent modified using amination to decrease the reactive step edge surface sites. Clearly, the modification increases the adsorbent's ability to desorb polar compounds. In the unmodified case, the largest percent recovery for these analytes is 17%, while the, modified sorbent yields as much as 97% recovery of the analytes.

### Example 2

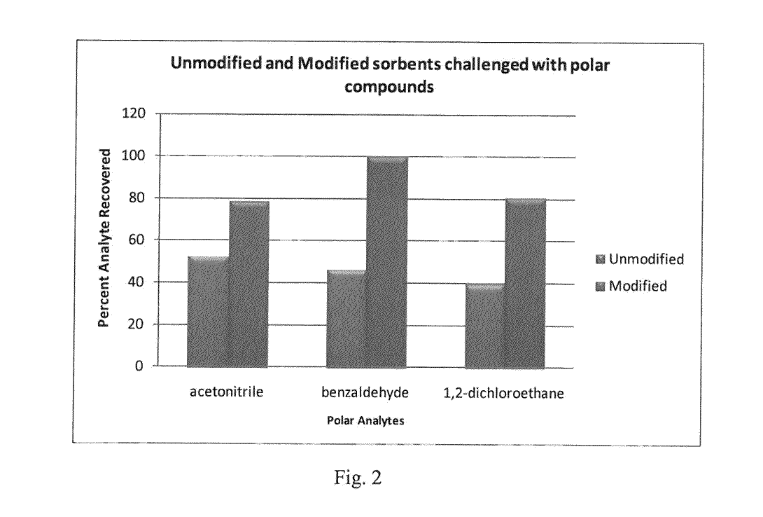

Results in FIG. 2 show the desorption results of an analyte mix with polar compounds from the unmodified sorbent and the modified aminated sorbent. Clearly, the modification increases the adsorbent's ability to desorb polar compounds. For each analyte, the analyte percent recovery from desorption is doubled or nearly doubled.

The present invention offers a way to provide enhanced adsorption via high surface area and mass transfer rates, and decreased reactivity at surface sites attractive to polar compounds. Taken together, these characteristics lead to less material incorporated into an environmental sampling device or chemical trapping system while increasing the fidelity and accuracy for identification of compounds in initial air samples.

What is claimed is:

1. A method for providing superadsorption of polar organic compounds using a material system comprising the steps of:
(a) enhancing adsorption by means of using high surface area and mass transfer rates and decreased reactivity at surface sites attractive to the polar compounds; and
(b) employing consequence management by maintaining a high rate of adsorptivity combined with high fidelity and accuracy of the material system.

2. A method for modifying a superadsorbent material for air sampling applications comprising:
submerging a superadsorbent material in a Schlenk flask, wherein the Schlenk flask contains a charged bath;
degassing the Schlenk flask;
heating the charged bath to a temperature;
removing the superadsorbent material from the charged bath;
washing the superadsorbent material; and
drying the superadsorbent material.

3. The method of claim 2, wherein the superadsorbent material is a carbide derived carbon.

4. The method of claim 2, wherein the degassing further comprises evacuating an amount of air from the Schlenk flask.

5. The method of claim 2, wherein washing is conducted using a solvent.

6. The method of claim 2, wherein drying is conducted in a vacuum environment for a time.

7. A modified superadsorbent material for air sampling applications comprising:
a superadsorbent material treated with a solution, thereby forming a treated superadsorbent material, wherein the treated superadsorbent material is substantially hydrophobic and is capable of adsorbing polar compounds.

8. The superadsorbent material of claim 7, wherein the superadsorbent material is a carbide-derived carbon.

9. The superadsorbent material of claim 7, wherein the polar compounds further comprise alcohols, amines, and hydrocarbons containing carboxyl groups.
